(19) United States
(12) Patent Application Publication
(10) Pub. No.: US 2009/0029891 A1
(43) Pub. Date: Jan. 29, 2009

(54) Soap device and method of combining pieces of soap

(76) Inventor: Matthew S. Callahan, Brighton, MA (US)

Correspondence Address: JAMES RAY & ASSOCIATES, 2640 PITCAIRN ROAD, MONROEVILLE, PA, 15146, US

(21) Appl. No.: 12/218,444

(22) Filed: Jul. 15, 2008

Related U.S. Application Data
(60) Provisional application No. 60/952,420, filed on Jul. 27, 2007.

(51) Int. Cl. A61K 8/02

(52) U.S. Cl. 510/146; 264/261

(57) ABSTRACT

A soap device is provided. Such soap device includes a soap bar member manufactured from a first predetermined material and having a first predetermined size and a first predetermined shape. A depressed area is operably disposed in such soap bar member at a predetermined location thereon for receiving and housing at least one predetermined piece of soap. The depressed area has a second predetermined size and a second predetermined shape.

### CROSS REFERENCE TO RELATED APPLICATION

This patent application is related to and claims priority from U.S. Provisional Patent Application Ser. Number 60/952,420 filed Jul. 27, 2007.

### FIELD OF THE INVENTION

The present invention relates, in general, to soap and, more particularly, this invention relates to a soap device for receiving for receiving and housing a smaller piece of soap and a method of combining pieces of soap.

### BACKGROUND OF THE INVENTION

Prior to the conception and development of the present invention, soap as is generally well known in the prior art, has been formed into bars. As the soap is used the bar of soap diminishes. As the soap bar becomes smaller and smaller, it is more difficult and less convenient to use. Sometimes, small pieces of soap are caught in drains causing such drains to slow or clog. To avoid clogs people often simply throw away the small pieces of soap thereby failing to maximize their soap purchases.

Specifically of interest to the present invention are the following: Smith, et al, U.S. Pat. No. 5,030,405, discloses a method and apparatus for combining soap segments, including providing a soap mold member formed with complementarily configured shells defining an internal cavity therewithin securable to an elongate handle to secure soap segments therewithin. The shells include apertures directed there through, whereupon positioning a plurality of soap segments within the cavity; the mold is positioned within a container. The container includes a sponge core defining a central cavity defined by a diameter substantially equal to that defined by the shells when secured together. The sponge core is saturated with water prior to insertion of the mold therewithin to permit continuous moistening of the segments contained within the mold when positioned within the cavity. The soap from the mold may be further directed within a press.

Manderson, U.S. Pat. No. 4,917,589, discloses a soap bar forming apparatus including a generally hollow housing having an aperture extending there through and a removable base. After soaking a plurality of soap particles, or chips, each of which is the residue of a substantially used soap bar, in water to allow the soap particles to become soft and pliable, the soap particles are placed in the housing with its base attached thereto. A plunger, or compressing element, is then inserted into the housing's aperture and manually displaced therein so as to compress the soap particles which adhere to one another in forming a composite bar of soap with a shape defined by the housing, its base, and the plunger.

Cerrone, U.S. Pat. No. 4,781,564, discloses a soap press component and a grinding component, operatively associated with one another. The grinding component has an inlet for receiving small soap bars or end pieces, and means for grinding them up to even finer pieces or shavings. The soap press component has wall structure defining a chamber having open top and bottom ends; a removable bottom wall is used to close the open bottom end, and a movable upper shaping form is used to close the open top end.

### SUMMARY OF THE INVENTION

The present invention provides a soap device. Such soap device includes a soap bar member manufactured from a first predetermined material and having a first predetermined size and a first predetermined shape. A depressed area is operably disposed in such soap bar member at a predetermined location thereon for receiving and housing at least one predetermined piece of soap. The depressed area has a second predetermined size and a second predetermined shape.

According to another embodiment, a method of combining pieces of soap is provided. Such method includes the steps of providing a bar of soap having a depressed area on a predetermined side thereof, applying water to at least one of such depressed area, at least one predetermined piece of soap, and a combination thereof for softening such at least one of such depressed area, such at least one predetermined piece of soap and such combination thereof, and inserting such at least one predetermined piece of soap into such depressed area.

### OBJECTS OF THE INVENTION

It is, therefore, one of the primary objects of the present invention to provide a device for enabling a user to use the small pieces of soap that are left over from larger bars of soap upon such larger bars being substantially used.

Another object of the present invention is to provide a method of combining pieces of soap such that small pieces of soap, left over from the use of a larger soap bar, will not be wasted.

Still another object of the present invention is to provide a device and a method of using such device which will enable a user to save money.

Yet another object of the present invention is to provide a device which can reduce the possibility of small soap pieces clogging a drain by providing a way to combine such small soap pieces with a larger soap bar.

In addition to the various objects and advantages of the present invention described with some degree of specificity above it should be obvious that additional objects and advantages of the present invention will become more readily apparent to those persons who are skilled in the relevant art from the following more detailed description of the invention, particularly, when such description is taken in conjunction with the attached drawing figures and with the appended claims.

### BRIEF DESCRIPTION OF THE DRAWINGS

FIG. 1 is a partial perspective view of the invention according to one embodiment of the invention illustrating such soap bar member having a depressed area on a predetermined side of the soap bar member.

FIG. 2 is a partial perspective view of the invention according to one embodiment of the invention in which a piece of soap has been placed within such depressed area of such soap bar member.

### BRIEF DESCRIPTION OF A PRESENTLY PREFERRED AND VARIOUS ALTERNATIVE EMBODIMENTS OF THE INVENTION

Prior to proceeding to the more detailed description of the present invention it should be noted that, for the sake of clarity and understanding, identical components which have identical functions have been identified with identical reference numerals throughout the several views illustrated in the drawing figures.

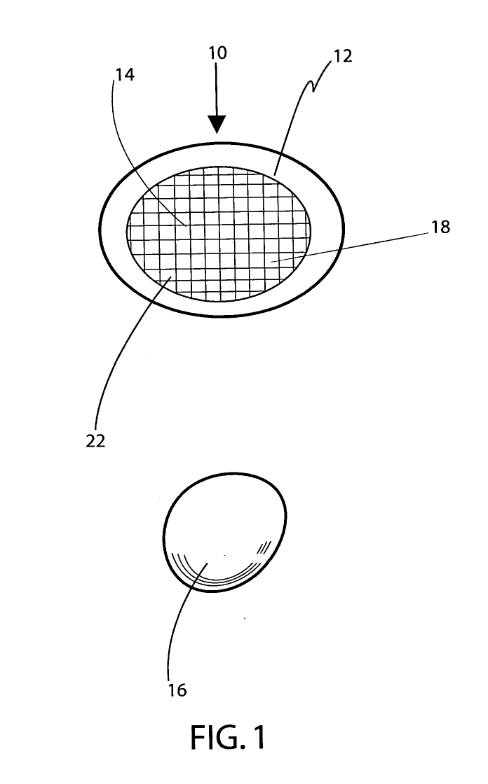

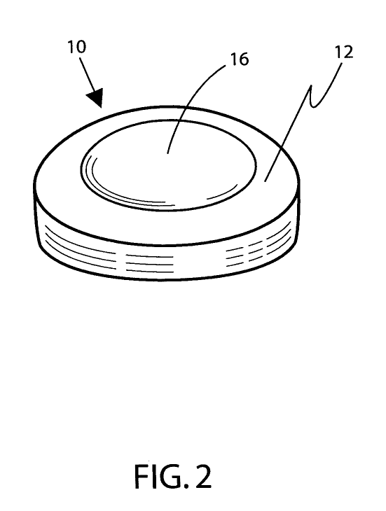

Reference is now made, more particularly, to FIGS. 1-2. A soap device, generally designated 10, is provided. Such soap device 10 includes a soap bar member 12 manufactured from a first predetermined material and having a first predetermined size and a first predetermined shape.

It is presently preferred that such soap bar member 12 includes at least one of a skin softening means, a scenting means, an antibacterial means, an exfoliating means, an acne controlling means, and a combination thereof.

A depressed area 14 is operably disposed in such soap bar member 12 at a predetermined location thereon for receiving and housing at least one predetermined piece of soap 16.

Such depressed area 14 has a second predetermined size and a second predetermined shape. Such depressed area 14 may be square, rectangular, rounded, oval shaped and any other shape which may reasonably facilitate its purpose.

It is presently preferred that such depressed area 14 further includes at least one textured surface 18 disposed on a predetermined location therein. It is presently preferred that such textured surface 18 includes at least one of grooves, indentations, striations, bumps, apertures, depressions, and a combination thereof.

It is presently preferred that such soap device 10 further includes a means 22 for securing such at least one predetermined piece of soap within such depressed area 14. Such means 22 may be such textured surface 18 gripping such at least one piece of soap 16 within such depressed area 14. However, such means is not meant to be limited as such and may also include wetting at least one of such depressed area 14, such at least one predetermined piece of soap 16, and a combination thereof such that such at least one predetermined piece of soap 16 sticks to such depressed area 14 thereby combining such at least one predetermined piece of soap 16 with such soap bar member 12.

According to another embodiment, a method of combining pieces of soap is provided. Such method includes providing a bar of soap having a depressed area on a predetermined side thereof. Applying water to at least one of such depressed area, at least one predetermined piece of soap, and a combination thereof for softening at least one of such depressed area, such at least one predetermined piece of soap and a combination thereof. Inserting such at least one predetermined piece of soap into such depressed area.

It is presently preferred that such method further includes at least one of molding such at least one predetermined piece of soap and applying pressure to such at least one predetermined piece of soap after such at least one predetermined piece of soap has been placed within such depressed area such that such at least one predetermined piece of soap is substantially flush with such soap bar member.

It is presently preferred that such method further includes texturing at least one surface disposed within such depressed area at a predetermined location therein.

It is further presently preferred that such method further includes providing at least one of grooves, indentations, striations, bumps, apertures, depressions, and a combination thereof on at least one surface disposed within such depressed area at a predetermined location thereon thereby forming such texturing.

While a presently preferred and various alternative embodiments of the present invention have been described in sufficient detail above to enable a person skilled in the relevant art to make and use the same it should be obvious that various other adaptations and modifications can be envisioned by those persons skilled in such art without departing from either the spirit of the invention or the scope of the appended claims.

I claim:

1. A soap device comprising
a. a soap bar member manufactured from a first predetermined material and having a first predetermined size and a first predetermined shape; and
b. a depressed area operably disposed in said soap bar member at a predetermined location thereon for receiving and housing at least one predetermined piece of soap, said depressed area having a second predetermined size and a second predetermined shape.

2. A soap device according to claim 1 wherein said depressed area further includes at least one textured surface disposed on a predetermined location therein.

3. A soap device according to claim 2 wherein said textured surface includes at least one of grooves, indentations, striations, bumps, apertures, depressions, and a combination thereof.

4. A soap device according to claim 1 wherein said soap bar member includes at least one of a skin softening means, a scenting means, an antibacterial means, an exfoliating means, an acne controlling means, and a combination thereof.

5. A soap device according to claim 1 wherein said soap device further includes a means for securing said at least one predetermined piece of soap within said depressed area.

6. A method of combining pieces of soap comprising the steps of:
a. providing a bar of soap having a depressed area on a predetermined side thereof;
b. applying water to at least one of said depressed area, at least one predetermined piece of soap, and a combination thereof for softening said at least one of said depressed area, said at least one predetermined piece of soap and said combination thereof; and
c. inserting said at least one predetermined piece of soap into said depressed area.

7. A method of combining pieces of soap according to claim 6 wherein said method further includes texturing at least one surface disposed within said depressed area at a predetermined location therein.

8. A method according to claim 7 wherein said method further includes providing at least one of grooves, indentations, striations, bumps, apertures, depressions, and a combination thereof on at least one surface disposed within said depressed area at a predetermined location thereon thereby forming said texturing.

9. A method according to claim 6 wherein said method further includes at least one of molding said at least one predetermined piece of soap and applying pressure to said at least one predetermined piece of soap after said at least one predetermined piece of soap has been placed within said depressed area such that said at least one predetermined piece of soap is substantially flush with said soap bar member.
